(19) United States
(12) Patent Application Publication
(10) Pub. No.: US 2025/0332119 A1
(43) Pub. Date: Oct. 30, 2025

(54) NOVEL ANTI-INFLAMMATORY COMPOSITION AND USE THEREOF

(71) Applicant: ASK CO., LTD., Daegu (KR)

(72) Inventors: Joong Hyun AHN, Daegu (KR); Dae Bang SEO, Yongin-si (KR); Han Woong PARK, Sejong-si (KR); Dae Sung YOO, Hwaseong-si (KR); Eun Jin SUNG, Gwangju-si (KR)

(73) Assignee: ASK CO., LTD., Daegu (KR)

(21) Appl. No.: 19/265,100

(22) Filed: Jul. 10, 2025

Related U.S. Application Data
(63) Continuation of application No. PCT/KR2023/021224, filed on Dec. 21, 2023.

(30) Foreign Application Priority Data
Jan. 13, 2023 (KR) 10-2023-0005504

(51) Int. Cl. A61K 31/047; A61P 29/00

(52) U.S. Cl. CPC A61K 31/047; A61P 29/00

(57) ABSTRACT

The present invention relates to a novel anti-inflammatory composition and a use thereof. More specifically, the present invention relates to an anti-inflammatory composition and a use thereof, the composition comprising araliadiol as an active ingredient in order to improve skin condition and prevent inflammatory skin diseases through an anti-inflammatory effect.

### CROSS-REFERENCE TO RELATED APPLICATIONS

This application is a bypass continuation of International Application No. PCT/KR2023/021224, filed on Dec. 21, 2023, which claims priority to Korean Patent Application No. 10-2023-0005504 filed Jan. 13, 2023, the entire disclosures of which are incorporated herein by reference.

### INCORPORATION BY REFERENCE OF SEQUENCE LISTING

The content of the electronically submitted sequence listing, file name: Q309396_sequence listing as filed.xml; size: 6,122 bytes; and date of creation: Jul. 10, 2025, filed herewith, is incorporated herein by reference in its entirety.

### TECHNICAL FIELD

The present invention relates to a novel anti-inflammatory composition and uses thereof. More specifically, the present invention relates to a composition for improving and preventing skin inflammation, comprising araliadiol or a pharmaceutically acceptable salt thereof as an active ingredient, and uses thereof.

### BACKGROUND ART

Inflammation is a normal biological reaction that occurs in response to harmful stimuli, such as physical injury or microbial infection. It is a protective response involving immune cells, blood vessels, and inflammatory mediators, which is one of the defense mechanisms in the body. The purpose of such an inflammatory response is to suppress cell damage at an early stage, to neutralize or eliminate pathogenic factors, and simultaneously, to regenerate damaged tissues to restore regular structures and functions.

Skin is the outermost organ of the human body and serves as an essential barrier that protects the body by retaining internal moisture and blocking invasion factors from the external environment. Factors that induce skin inflammation include physicochemical stimuli, allergens, ultraviolet (UV), oxidative stress and pathogenic infections, as well as inflammation-related cytokines and chemokines that are produced accordingly. The general pathway of skin inflammatory responses is an immune response aimed at protecting the body from external stimuli, and the suppression of the inflammatory response is a major therapeutic target for the control of inflammation.

When an inflammatory response occurs, inflammatory factors such as nitric oxide and prostaglandin E2 are produced along with free radicals. Free radicals are generally involved in maintaining homeostasis in terms of cell differentiation, growth, and survival. However, reactive oxygen species (ROS) among free radicals are continuously generated by the oxidation and reduction of oxygen mediated by way of respiration and immune responses. Due to their reactive nature, ROS can exert harmful effects on the body and are therefore eliminated through the body's antioxidant mechanisms. However, if the balance between the generation and elimination of ROS is disrupted, oxidative stress may occur causing problems such as inflammation, aging, tissue damage, and cancer. For this reason, it is necessary to reduce inflammation by administering substances with anti-inflammatory activity to the body, skin, etc.

Since most inflammation treatments are obtained through synthesis, efforts are required to treat inflammation using substances that can be obtained from natural resources, which are relatively safe for the human body. In particular, materials derived from plants have long been used due to their excellent safety. Especially in Korea, the development of functional materials primarily based on plants and herbal ingredients used in folk remedies or Korean traditional medicines has been actively carried out.

Centella asiatica L. is a perennial creeping plant belonging to the Apiaceae family that is tasteless and odorless. It grows extensively in hot and humid regions, including the Indian Ocean, southern India, and Malaysia. In Korea, it is known to grow in clusters in low-humid areas such as Jeju Island and certain southern regions. Centella asiatica has long been widely used as a medicinal plant for traditional medicines in India and other parts of Asia, and particularly contains main ingredient such as madecassoside, madecassic acid, asiaticoside and asiatic acid, which exhibit various effects, including antioxidant, anti-cancer, collagen synthesis-promoting, memory-enhancing, brain neuroprotective, and anti-allergic effects. Accordingly, it has been used not only as a raw material in pharmaceuticals for treatment and prevention, but also as an ingredient for cosmetics.

Araliadiol, a polyacetylene compound, is a natural substance primarily obtained from extracts of plants such as Aralia elata and Centella asiatica. Araliadiol has been reported to inhibit the growth of human breast carcinoma cells (MCF-7) (Planta Med. 2011; 77 (2): 164-8) or to effectively enhance glucose uptake by skeletal muscle cells (J Biotechnol. 2023; 368:53-59). However, to the best knowledge of the inventors, the anti-inflammatory activity of araliadiol has not been studied or known.

### PRIOR ART LITERATURE

### Non-Patent Literature

(0001) Cheng et al., Inhibitory effect of human breast cancer cell proliferation via p21-mediated G1 cell cycle arrest by araliadiol isolated from Aralia cordata Thunb. Planta Med. 2011; 77 (2): 164-8.
(0002) Jo et al., Polyacetylenes from the adventitious roots of Centella asiatica with glucose uptake stimulatory activity. J Biotechnol. 2023; 368:53-59.

### DETAILED DESCRIPTION

### Technical Objective

The objective of the present invention resides in providing a composition comprising plant-derived natural materials with various efficacies as main ingredients, which has the excellent effect of improving skin inflammation.

Specifically, the objective of the present invention resides in providing a composition comprising an anti-inflammatory ingredient for reducing the production of proinflammatory cytokines and nitric oxide.

Another objective of the present invention resides in providing a cosmetic composition comprising an anti-inflammatory ingredient for alleviating skin inflammation.

Another objective of the present invention resides in providing a pharmaceutical composition for the preventing or treating inflammatory skin diseases, comprising an anti-inflammatory ingredient.

Another objective of the present invention resides in providing a food composition comprising an anti-inflammatory ingredient.

However, the objectives of the present invention are not limited to these, and other unmentioned objectives and advantages of the present invention can be understood from the following descriptions and will be more clearly understood by the embodiments of the present invention. Furthermore, the objectives and advantages of the present invention can be readily realized by the means disclosed in the claims and the combinations thereof.

### Means for Solving the Problems

The anti-inflammatory composition of the present invention for achieving the aforementioned objectives is characterized by comprising araliadiol or a pharmaceutically acceptable salt thereof.

In one embodiment of the present invention, the anti-inflammatory composition comprising araliadiol or a pharmaceutically acceptable salt thereof may be characterized by reducing inflammatory activity. Specifically, the anti-inflammatory composition of the present invention may be characterized by having any one of the activities among reduction in NO production, reduction in PGE2 production, reduction in IL-1α expression, and reduction in TNF-α expression.

In one embodiment of the present invention, the present invention is characterized by a cosmetic composition for preventing dermatitis and improving skin condition, comprising araliadiol or a pharmaceutically acceptable salt thereof.

In one embodiment of the present invention, the present invention is characterized by a pharmaceutical composition for the preventing or treating inflammatory skin diseases, comprising araliadiol or a pharmaceutically acceptable salt thereof.

In one embodiment of the present invention, the present invention is characterized by a food composition for anti-inflammatory purposes, comprising araliadiol or a pharmaceutically acceptable salt thereof.

### Working Effect of the Invention

The present invention relates to an anti-inflammatory composition comprising araliadiol or a pharmaceutically acceptable salt thereof as an active ingredient. The anti-inflammatory composition according to the present invention demonstrates remarkable effects in reducing the production of nitric oxide (NO) and prostaglandin E2 (PGE2), the expression of interleukin-1α (IL-1α) and tumor necrosis factor-α (TNF-α). As such, the composition of the present invention can improve skin conditions caused by inflammation and prevent or treat inflammatory skin diseases.

### BRIEF DESCRIPTION OF DRAWINGS

FIGS. 1a and 1b represent a graph showing NO production, cell viability (FIG. 1a) and Realtime qRT-PCR results (FIG. 1b) of the primary fraction samples of Centella asiatica extract of the BT-Care variety.

FIGS. 2a and 2b represent a graph showing NO production, cell viability (FIG. 2a) and Realtime qRT-PCR results (FIG. 2b) of the secondary fraction samples of Centella asiatica extract of the BT-Care variety.

FIG. 3a represents a graph showing NO production, cell viability and Realtime qRT-PCR results of the third fraction samples of Centella asiatica extract of the BT-Care variety.

FIG. 3b and FIG. 3c represent graphs showing the results of Realtime qRT PCR (FIG. 3b) and ELISA (FIG. 3c) of Assay-5 and Assay-10 of a third fraction sample from a Centella asiatica extract of the BT-Care variety.

FIG. 4 shows the results of ODS TLC (octadecylsilicate thin layer chromatography) performed on Assay-5 and Assay-10 together with asiatic acid and madecassic acid in 90% methanol.

FIGS. 5a and 5b show the results of molecular weight analysis of Assay-5 (FIG. 5a) and Assay-10 (FIG. 5b).

FIGS. 6a and 6b show the results of 1H NMR spectrum (FIG. 6a) and 13C NMR spectrum (FIG. 6b) measurements of Assay-5.

FIGS. 7a and 7b show the results of 1H NMR spectrum (FIG. 7a) and 13C NMR spectrum (FIG. 7b) measurements of Assay-10.

FIGS. 8a to 8c show the results of 1H-1H COSY spectrum (FIG. 8a), HMQC spectrum (FIG. 8b), and HMBC spectrum (FIG. 8c) measurements of Assay-5.

FIGS. 9a-9c shows the results of 1H-1H COSY spectrum (FIG. 9a), HMQC spectrum (FIG. 9b), and HMBC spectrum (FIG. 9c) measurements of Assay-10.

FIGS. 10a and 10b shows the chemical structures of Assay-5 (FIG. 10a) and Assay-10 (FIG. 10b).

FIG. 11 is a comparison table of the whole plant and the individual parts (basal leaf, rhizome, and cauline leaf) of Centella asiatica from the ‘BT-Care’ variety and a common variety (Tiger Care).

### MODE FOR CARRYING OUT THE INVENTION

Hereinafter, the present invention is described in detail. However, in describing the present invention, detailed descriptions regarding known related technologies may be omitted when they would obscure the essence of the present invention.

The terms or words used in this specification and the claims shall be construed to have meanings and concepts consistent with the technical idea of the present invention, based on the principle that an inventor may define terms or words to best describe his or her invention. Furthermore, the embodiments described herein and the configurations shown in the drawings are only one embodiment in which the present invention may be implemented and do not represent the entirety of the technical idea of the invention. Therefore, it should be understood that various equivalents, modifications, and applicable examples capable of replacing them at the time of filing may exist. In addition, each description and embodiment disclosed in the present invention may be applied to other descriptions and embodiments, and all combinations of various elements disclosed in the present invention fall within the scope of the invention, and the scope of the present invention should not be construed as being limited by the specific descriptions set forth below.

The anti-inflammatory composition for providing the effect of improving and preventing skin inflammation according to the present invention is characterized by comprising araliadiol or a pharmaceutically acceptable salt thereof.

In one embodiment of the present invention, the anti-inflammatory composition comprising araliadiol or a pharmaceutically acceptable salt thereof may alleviate inflammation by reducing prostaglandin E2 (PGE2) and nitric oxide (NO), generated by the activity of pro-inflammatory cytokines and macrophage.

In the present invention, the formation of pro-inflammatory cytokines such as IL-1α and TNF-α results in the conversion of arachidonic acid into prostaglandins through the activation of phospholipase A2 and the generation of nitric oxide (NO). In addition, macrophages release reactive oxygen from their cell membranes to dissolve foreign substances in order to phagocytose bacteria or fungi that have invaded from the outside. However, when the activity of macrophages continues to increase, leading to more frequent reactions and elevated secretion of reactive oxygen, the reactive oxygen may leak out of the cell and attack surrounding blood vessel walls or tissues, thereby causing disease. Prostaglandin E2 is also an inflammatory factor generated by the activity of macrophages, and it causes symptoms such as vasodilation, bronchodilation, inhibition of gastric acid secretion, inflammation, and fever.

In one embodiment of the present invention, araliadiol exhibits higher anti-inflammatory activity than linoleic acid. Linoleic acid is a polyunsaturated omega-6 fatty acid and one of the essential fatty acids for humans that must be obtained through diet. Linoleic acid is widely used as a cosmetic composition due to its various activities, including alleviating skin pigmentation (Arch Dermatol Res. 1998; 290 (7): 375-81), strengthening the skin barrier (Acta Paediatr. 2002; 91 (5): 546-54) and reducing the size of microcomedones (Clin Exp Dermatol. 1998; 23 (2): 56-8) due to the inhibition of melanin production by active melanocytes and the enhancement of the desquamation of melanin pigments in the epidermis. In addition, it is known to exhibit anti-inflammatory activity by inhibiting NO production in macrophages and suppressing the expression of proinflammatory cytokines including TNF-α, IL-6, IL-1B, and NOS2, thereby reducing inflammatory activity (Food Funct. 2017; 8 (11): 4150-4158).

The extraction methods for obtaining the Centella asiatica extract may be prepared according to any known extraction methods commonly used in the technical field to which the present invention belongs. More specifically, conventionally used extraction methods such as cold maceration, heated extraction, ultrasonic extraction, filtration, pressurized extraction, reflux extraction, supercritical extraction, electrical extraction, etc. may be used. Further, conventional extractors, ultrasonic extractors or fractionators may be used.

In one embodiment of the present invention, the anti-inflammatory composition comprising araliadiol or a pharmaceutically acceptable salt thereof may be used in a cosmetic composition in order to be provided in a suitable form to consumers who desire anti-inflammatory effect to improve skin conditions caused by inflammation and to prevent inflammatory skin diseases.

In the present invention, “improvement” refers to all actions that heal skin aging and damage, or, even without complete healing, suppress the progression or worsening of symptoms to stop advancement of damage, or guide some or all symptoms toward healing.

The cosmetic composition may be prepared in any formulation known in the art. For example, it may be formulated as a solution, suspension, paste, gel, cream, lotion, powder, soap, surfactant-containing cleanser, oil, powder foundation, emulsion foundation, wax foundation, or spray. More specifically, it may be prepared as formulations such as softening toner, nourishing toner, nourishing cream, massage cream, essence, eye cream, cleansing cream, cleansing foam, cleansing water, pack, spray or powder, water-in-oil type, oil-in-water type formulation, or ointment.

In addition, the cosmetic composition may further comprise functional additives and ingredients included in general cosmetic compositions, in addition to the active ingredients disclosed in the present specification. Specifically, the composition may further comprise conventionally used purified water, thickeners, preservatives, stabilizers, solubilizers, surfactants, carriers, fragrances, or combinations thereof. The functional additives may comprise ingredients selected from the group consisting of water-soluble vitamins, fat-soluble vitamins, polymeric peptides, polymeric polysaccharides, sphingolipids, and seaweed extracts. The carriers may be, for example, alcohols, oils, surfactants, fatty acids, silicone oils, humectants, moisturizers, viscosity modifiers, emulsions, stabilizers, ultraviolet scatterers, ultraviolet absorbers, colorants, fragrances, and the like. Since the compounds or compositions that may be used as alcohols, oils, surfactants, fatty acids, silicone oils, humectants, moisturizers, viscosity modifiers, emulsions, stabilizers, ultraviolet scatterers, ultraviolet absorbers, colorants, and fragrances are well known in the art, those skilled in the art can select and use suitable substances or compositions. In addition, the cosmetic composition may further comprise, if necessary, sunscreens, antioxidants (butylhydroxyanisole, propyl gallic acid, erythorbic acid, tocopheryl acetate, butylated hydroxytoluene, etc.), preservatives (methylparaben, butylparaben, propylparaben, phenoxyethanol, imidazolidinyl urea, chlorphenesin, etc.), colorants, pH regulators (triethanolamine, citric acid, citric acid, sodium citrate, malic acid, sodium malate, fumaric acid, sodium fumarate, succinic acid, sodium succinate, sodium hydroxide, sodium dihydrogen phosphate, etc.), moisturizers (glycerin, sorbitol, propylene glycol, butylene glycol, hexylene glycol, diglycerin, betaine, glycereth-26, methyl gluceth-20, etc.), lubricants and the like.

In each formulation of the cosmetic composition, appropriate ingredients may be selected and blended according to the formulation or intended purpose of the cosmetic. The blending ingredients and methods may be carried out using conventional techniques in the art.

In the present invention, an “inflammatory skin disease” may refer to any one selected from the group consisting of dermatitis, allergic dermatitis, irritant dermatitis, seborrheic dermatitis, atopic dermatitis, sensitive dermatitis, pruritus, eczematous skin disease, xerotic eczema, erythema, urticaria, psoriasis, drug eruption, acne, and vaginitis.

In one embodiment of the present invention, an anti-inflammatory composition comprising araliadiol or a pharmaceutically acceptable salt thereof may be used as a pharmaceutical composition for the prevention or treatment of inflammatory skin diseases.

In the present invention, “pharmaceutical composition” may refer to a molecule or compound that confers one or more beneficial effects upon administration to a subject. The beneficial effects may include enabling diagnostic determination; improvement of diseases, symptoms, disorders, or conditions; reducing or preventing the onset of diseases, symptoms, disorders, or conditions; and responding diseases, symptoms, disorders, or conditions in general.

In the present invention, “treatment” refers to any actions by which diseases, disorders, or their attendant symptoms are improved or beneficially altered.

In the present invention, “prevention” collectively refers to partially or completely delaying or preventing the onset or recurrence of a disease, disorder, or its attendant symptoms, preventing the acquisition or reacquisition of diseases or disorders, or reducing the risk of acquiring diseases or disorders. The prevention refers to any actions of inhibiting or delaying the onset of skin-damaging diseases, disorders, or symptoms by using a composition according to one embodiment of the present invention.

The pharmaceutical composition may be formulated as a preparation selected from the group consisting of tablets, soft or hard capsules, pills, powders, suspensions, syrups, injections, and granules.

The pharmaceutical composition may be intended for oral or parenteral administration.

The concentration of the active ingredient of the pharmaceutical composition, based on the total weight of the composition, may be from 0.00001 to 9 wt %, for example 0.00001 wt % or more, 0.0001 wt % or more, 0.001 wt % or more, 0.01 wt % or more, 9 wt % or less, 8 wt % or less, 7 wt % or less, 6 wt % or less, 5 wt % or less, 4 wt % or less, 3 wt %, 2 wt % or less, or 1 wt % or less. In one embodiment of the present specification, the concentration of the active ingredient may preferably be from 0.0001 to 5 wt %, and more preferably from 0.001 to 3 wt %.

The pharmaceutical composition may comprise conventional excipients such as fillers, extenders, binders, disintegrants, anticoagulants, lubricants, humectant, pH regulators, nutrients, vitamins, electrolytes, alginic acid and salts thereof, pectic acid and salts thereof, protective colloids, glycerin, flavorings, emulsifiers, or preservatives, and the like.

The formulation of the pharmaceutical composition may vary depending on the method of administration and may be formulated using methods well known in the technical field of the present invention to provide rapid, sustained, or delayed release of the active ingredient after administration to a mammal.

One embodiment of the invention provides a method for preventing, improving, or treating a condition in a subject, comprising the step of administering the pharmaceutical composition to the subject.

The condition of the subject may be related to skin or may be related to inflammation.

Administration may be performed by any method known in the art. The composition may be administered directly to the subject by routes such as intravenous, intramuscular, oral, transdermal, mucosal, intranasal, intratracheal, or subcutaneous administration. The administration may be systemic or localized.

The subject may be a mammal, for example, a human, cow, horse, pig, dog, sheep, goat, or cat. The subject may be one in need of improvement in skin condition or treatment of an inflammatory skin diseases.

The administration may be carried out by administering the composition according to one embodiment in an amount of 0.00001 mg to 1,000 mg per subject per day, for example, 0.00001 mg to 500 mg, 0.00001 mg to 100 mg, 0.00001 mg to 50 mg, 0.00001 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg. However, the dosage may be prescribed variably depending on factors such as the method of formulation, mode of administration, age, body weight, sex, pathological condition, diet, time of administration, route of administration, rate of excretion, and response sensitivity of the patient. A person skilled in the art can adjust the dosage appropriately in view of these factors. The frequency of administration may be once or two or more times daily within a range of clinically acceptable side effects, and the administration may be conducted to one or two or more sites, with the total number of administration days per treatment ranging from 1 to 30 days, with administrations given daily or at intervals of 2 to 5 days. If necessary, the same treatment may be repeated after an appropriate period of time. For animals other than humans, the same dose per kg as for humans may be administered, or for example, the above dosages may be administered in an amount obtained by converting the above amount in consideration of the volume ratio (e.g., average value) of the target animal and human organ (e.g., heart).

In one embodiment of the present invention, an anti-inflammatory composition comprising araliadiol or a pharmaceutically acceptable salt thereof may be used as an anti-inflammatory food composition. In such cases, the anti-inflammatory food composition of the present invention may be appropriately used by conventional methods, such as being added to a food product as is or in combination with other food products or food ingredients. Typically, in the preparation of a food or beverage, the anti-inflammatory food composition of the present invention is added to raw materials in an amount of 15 parts by weight or less, preferably 10 parts by weight or less. However, in the case of long-term consumption for health purposes, the food composition may be added in an amount less than the above range.

There is no particular restriction on the type of food products to which the anti-inflammatory food composition is added. Examples include meat, sausages, bread, chocolate, candy, snacks, confectionery, pizza, ramen, other noodles, chewing gum, dairy products including ice cream, various soups, beverages, tea drinks, alcoholic beverages, and vitamin complexes, encompassing all food products in the conventional sense.

Food products comprising the anti-inflammatory food composition of the present invention may further comprise ingredients conventionally used in food manufacturing, such as proteins, carbohydrates, fats, nutrients, seasonings, and flavoring agents. Examples of carbohydrates as described above include monosaccharides, such as glucose and fructose; disaccharides, such as maltose, sucrose and oligosaccharides; polysaccharides, such as dextrins and cyclodextrins, which are conventional sugars, and sugar alcohols, such as xylitol, sorbitol and erythritol. As flavoring agents, natural ones, such as thaumatin and stevia extracts (for example, rebaudioside A and glycyrrhizin), and synthetic ones, such as saccharin and aspartame, may be used.

Food products comprising the anti-inflammatory food composition of the present invention may further comprise various nutrients, vitamins, electrolytes, flavoring agents, coloring agents, pectic acid and salts thereof, alginic acid and salts thereof, organic acids, protective colloidal thickeners, pH adjusters, stabilizers, preservatives, glycerin, alcohol, and carbonating agents used in carbonated beverages. The proportion of these additional ingredients is not particularly critical and is generally selected within the range of 0.01 to 1 part by weight based on 100 parts by weight of the anti-inflammatory food composition of the present invention.

In one embodiment of the present invention, araliadiol of the present invention may be a compound isolated from an extract of the Centella asiatica variety ‘BT-Care’ but is not limited thereto. ‘BT-Care’ refers to a new Centella asiatica variety filed with the Korea Forest Service under Variety Protection Application No. 2019-14. Compared to the control variety ‘Tiger Care’, ‘BT-Care’ exhibits greater length and width in characteristics such as the shape, length, and width of the leaf blade of a normal leaf, the diameter of the petiole of a normal leaf, and the length and diameter of the peduncle. In recognition of such uniformity and stability, it was finally registered under Variety Protection Registration No. 288.

Hereinafter, the present invention will be described in more detail with reference to embodiments. However, the following embodiments are provided for illustrative purposes only and are not intended to limit the scope of the present invention. For example, araliadiol used to achieve the objectives of the present invention is not limited to that obtained by the methods described in the following embodiments, and any araliadiol obtainable by known methods may be used to practice the present invention.

### Experimental Example 1. Test of Anti-Inflammatory Effect Using Realtime qRT-PCR

To evaluate the inhibitory ability against inflammatory cytokines induced by LPS (lipopolysaccharide), mouse macrophages were used to assess the effect on the production of IL-1α and TNF-α, which are the early-stage inflammatory molecules. The cells used in the experiment were RAW264.7 (Mouse macrophage), which were cultured at 37° C. under 5% CO₂ in Dulbecco's modified Eagle's medium (DMEM, Invitrogen) supplemented with 10% fetal bovine serum (FBS, Lonza) and 50 μg/mL streptomycin (Sigma).

Specifically, the cells were seeded into 6-well plates at a density of 1×10⁶ cells/well and cultured at 37° C. in a 5% CO₂ incubator for 24 hours. After removing the culture medium, the cells were replaced with fresh medium containing the sample and pre-incubated at 37° C. under 5% CO₂ for 30 minutes. Following pretreatment with the sample for 30 minutes, the cells were exposed with LPS for 24 hours to induce an inflammatory response. As a control, cells treated with LPS alone, without sample treatment, were used.

Total RNA in cells was then extracted from cultured cells using Trizol reagent (Invitrogen, USA) for RNA analysis. The purity and integrity of the RNA were confirmed by measuring the A260 nm/A280 nm ratio, and RNA yield was measured by absorbance at 260 nm.

For cDNA synthesis, 3 μg of total RNA was mixed with Oligo dT 15 (500 ng/μL) primer, dNTP (10 mM), RTase inhibitor (40 U/μL), and Powerscript II RTase (Clontech, USA), followed by primer annealing at 25° C. for 10 minutes, cDNA synthesis at 42° C. for 60 minutes, and RTase denaturation at 95° C. for 5 minutes. PCR was performed to amplify IL-1α, TNF-α, and GAPDH from cDNA by mixing 3 μL of cDNA, 5 μL of 10×taq polymerase buffer, 2 μL of 10 mM dNTP, 2 μL each of 10 pmol primers, and 0.5 μL of taq polymerase, followed by the addition of distilled water to a final volume of 50 μL.

The primer sequences used are shown in Table 1, and the PCR process was carried out using the Real-time qRT PCR method with the LightCycler® 96 System (Roche Diagnostics).

TABLE 1

| Gene | Primer |
|---|---|
| GAPDH | F: 5′ - AAC GAA TTT GGT CGA ACA GC - 3′ |
| | (SEQ ID NO: 1) |
| | R: 5′ - TGA GGA GGG ATT CAG TG - 3′ |
| | (SEQ ID NO: 2) |
| IL-1α | F: 5′ - TCA AGA TGG CCA AAG TTC CT - 3′ |
| | (SEQ ID NO: 3) |
| | R: 5′ - TGC AAG TCT CAT GAA GTG AGC - 3′ |
| | (SEQ ID NO: 4) |
| TNF-α | F: 5′ - CAA AGT AGA CCT GCC CAG AC - 3′ |
| | (SEQ ID NO: 5) |
| | R: 5′ - GAC CTC TCT CTA ATC AGC CC - 3′ |
| | (SEQ ID NO: 6) |

### Experimental Example 2. Test of Antioxidant and Anti-Inflammatory Effects Using NO & PGE2 ELISA Assays

To evaluate the inhibitory ability against nitric oxide and PGE2 production induced by LPS (lipopolysaccharide), mouse macrophages were used to assess the effect on the production of nitric oxide (NO) and prostaglandin E2 (PGE2). The cells used in the experiment were RAW264.7 (mouse macrophage), which were cultured in Dulbecco's modified Eagle's medium (DMEM, Invitrogen) supplemented with 10% fetal bovine serum (FBS, Lonza) and 50 μg/mL streptomycin (Sigma) at 37° C. in 5% CO₂.

Specifically, the cells were seeded into 96-well plates at a density of 1× 10⁵ cells/well and incubated at 37° C. in a 5% CO₂ incubator for 24 hours. The culture medium was removed from the cultured cells, and the medium was replaced with a fresh medium containing the test sample and the cells pre-incubated at 37° C. under 5% CO₂ for 30 minutes. Following the 30-minute incubation, LPS was added to a final concentration of 1 μg/mL, and the cells were incubated at 37° C. in a 5% CO₂ incubator for 24 hours. After incubation, 100 μL of the supernatant was transferred to a new 96-well plate and mixed with an equal volume of a 1:1 mixture of Griess reagent A and B, then 100 μL aliquot of the resulting mixture was dispensed into each well. Immediately after treatment with Griess reagent, absorbance was measured at 540 nm using a microplate reader. The NO production rate (%) of macrophages was calculated using Equation 1 below.

$$NO production ( \% ) = Absorbance of sample / Absorbance of control \times 100 \qquad [ Equation 1 ]$$

In addition, to evaluate the production of PGE2, a procedure similar to that described above was followed. The procedures for treating the cells with the sample and LPS after cell culture were identical. Subsequently, 100 μL aliquots of the supernatant were collected and transferred to a new 96-well plate, and diluted 10- to 20-fold with culture medium. In the NSB (Non-specific binding) wells of the Anti-Mouse IgG plate, 100 μL of culture medium was added. Then, 100 μL each of the standard and diluted supernatant were added to the respective wells, followed by 50 μL of blue PGE2-AP conjugate added to all wells except the blank well. 50 μL of culture medium was added to the NSB wells, and 50 μL of yellow PGE2 antibody was added to all wells except the blank and NSB wells. The plate was then shaken using an orbital shaker at 500 rpm for 2 hours at room temperature (22-25° C.). After 2 hours, the contents of the plate were discarded, and the wells were washed three times with 400 μL of washing buffer. Then, 200 μL of substrate solution was added to each well and the plate was incubated at room temperature (22-25° C.) for 45 minutes. After incubation, 50 μL of stop solution was added to each well, and the absorbance was immediately measured at 405 nm using a microplate reader. The PGE₂ production rate (%) of macrophages was calculated using Equations 2 and 3 below.

$$\% bound = [ ( Absorbance of sample - Absorbance of NSB ) / ( Absorbance of Blank - Absorbance of NSB ) ] \times 100 \qquad [ Equation 2 ]$$

$$( Standard curve = using the natural logarithm , \\ and the equation y = A * ln ( x ) + B is obtained . )$$

$${PGE}_{2} production = dilution factor \times ( e ( \% bound - B ) ) / A \qquad [ Equation 3 ]$$

### Example 1. Isolation and Purification of Active Ingredients from Centella Asiatica Extract

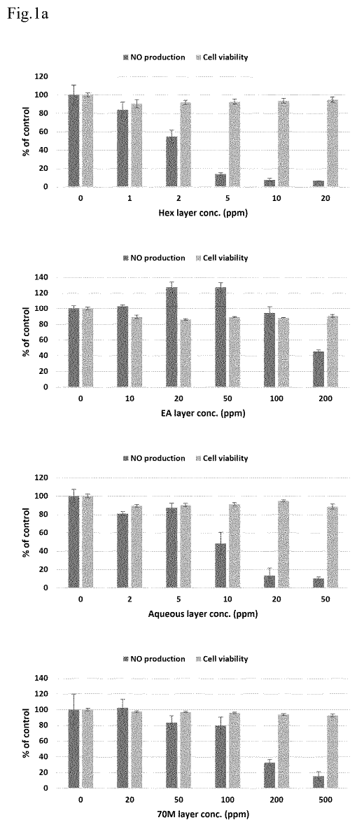

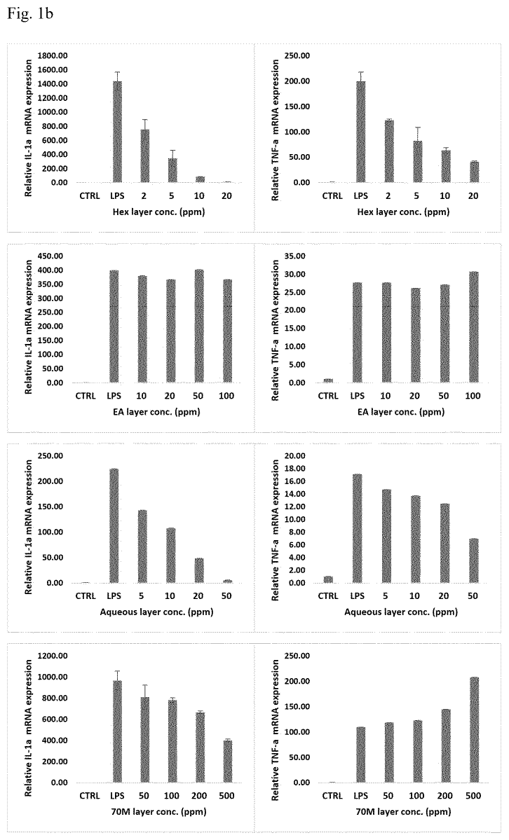

The Centella asiatica variety ‘BT-Care’ was extracted by adding the variety to an aqueous 70% methanol solution and concentrating the extract under reduced pressure to remove methanol. Hexane was added to the concentrated extract for liquid-liquid extraction, and the mixture was separated into a hexane layer and a water layer. The anti-inflammatory efficacy of the 70% methanol extract (70M layer), the hexane layer (Hex layer) of 70% methanol extract, the ethyl acetate layer (EA layer) of 70% methanol extract, and the aqueous layer (Aqueous layer) of 70% methanol extract obtained from the extract were evaluated, and the results are shown in FIGS. 1a and 1b, respectively.

Specifically, except for the ethyl acetate layer of the 70% methanol extract, the samples reduced NO production in a concentration-dependent manner within the concentration range maintaining over 80% cell viability (FIG. 1a) and also reduced the expression of Relative IL-1α mRNA and Relative TNF-α mRNA. However, the 70% methanol extract increased the expression of Relative TNF-α mRNA in a concentration-dependent manner (FIG. 1b). Since TNF-α is a factor that regulates immune cells, these results suggest that the 70% methanol extract enhances immunomodulatory activity in a concentration-dependent manner.

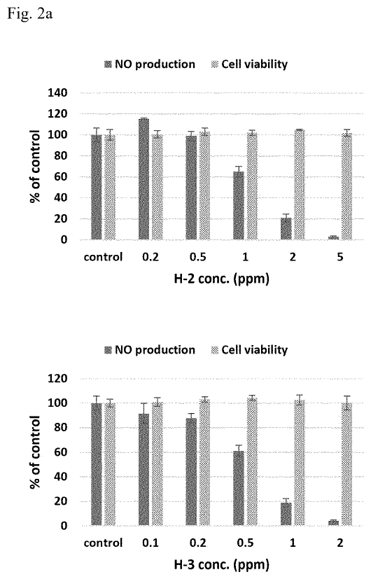

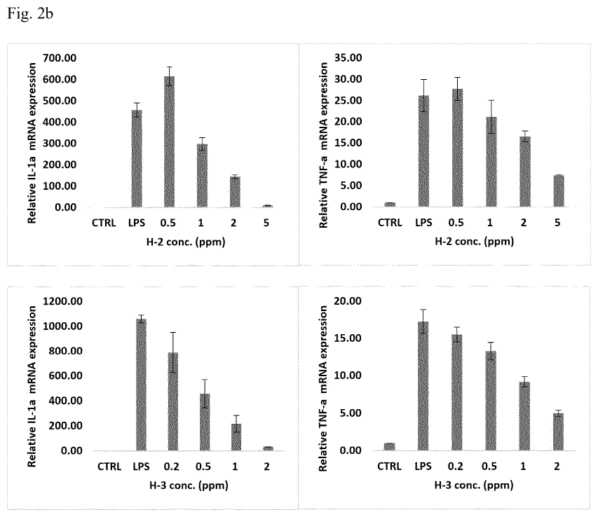

Based on the above results, the hexane layer was concentrated and subjected to flash silica gel column chromatography (eluent: hexane-ethyl acetate=20:1-1:1, v/v). Among these fractions, the hexane-ethyl acetate (10:1 and 5:1) fractions were further subjected to silica gel column chromatography (eluent: hexane-ethyl acetate=20:1-5:1, v/v) to obtain the active H-2 fraction. Furthermore, the hexane-ethyl acetate (2:1, v/v) fraction was further subjected to silica gel column chromatography (eluent: hexane-ethyl acetate=5:1-1:1, v/v) to obtain the active H-3 fraction. The anti-inflammatory efficacy of H-2 and H-3 fractions were evaluated, and the results are shown in FIGS. 2a and 2b, respectively.

For both H-2 and H-3, NO production decreased with increasing concentration (FIG. 2a), and IL-1α and TNF-α mRNA expression also decreased with increasing concentration (FIG. 2b).

H-2 was subjected to ODS MPLC (referring to medium pressure liquid chromatography using octadecylsilane as the stationary phase) (eluent: 0-100% aqueous methanol) and separated into five fractions: H2-1, H2-2, H2-3, H2-4, and H2-5. From fraction H2-1, the compound assay-1 (H212-2) was isolated by performing Sephadex LH-20 column chromatography (eluent: chloroform-methanol=1:1, v/v), followed by ODS MPLC (eluent: 50-100% aqueous methanol). From fraction H2-2, the compounds assay-2 (H222-1), assay-3 (H222-3), and assay-4 (H222-4) were purified by performing Sephadex LH-20 column chromatography (eluent: chloroform-methanol, 1:1, v/v), followed by ODS MPLC (eluent: 60-100% aqueous methanol). From fraction H2-3, the compound assay-5 (H232-3) was isolated by performing Sephadex LH-20 column chromatography (eluent: chloroform-methanol=1:1, v/v), followed by ODS MPLC (eluent: 70-100% aqueous methanol). From fraction H2-4, the compound assay-6 (H242-1) was purified by performing Sephadex LH-20 column chromatography (eluent: chloroform-methanol=1:1, v/v), followed by ODS MPLC (eluent: 70-100% aqueous methanol). From fraction H2-5, the compounds assay-7 (H25-1), assay-8 (H25-2), and assay-9 (H25-3) were isolated by performing Sephadex LH-20 column chromatography (eluent: chloroform-methanol=1:1, v/v).

Fraction H-3 was subjected to ODS MPLC (elution eluent: 0-100% aqueous methanol) and fractioned into nine groups H3-1, H3-2, H3-3, H3-4, H3-5, H3-6, H3-7, H3-8, and H3-9. From the most abundant fraction H3-1, the compound assay-10 (H312-1) was isolated by performing Sephadex LH-20 column chromatography (eluent: chloroform-methanol=1:1, v/v), followed by ODS MPLC (eluent: 50-100% aqueous methanol).

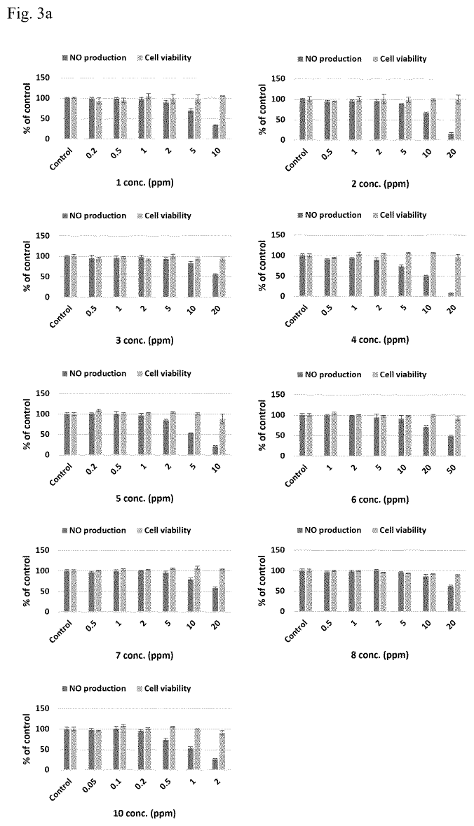

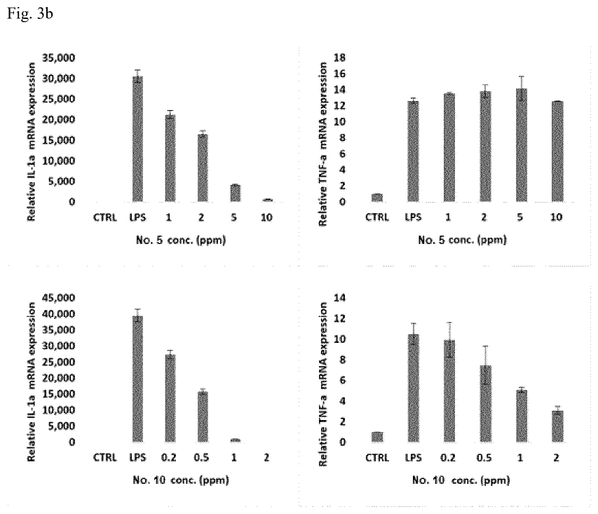

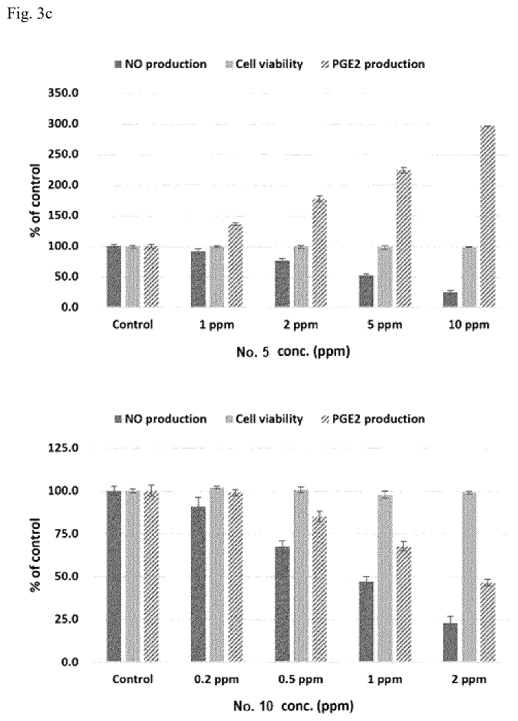

The anti-inflammatory efficacy of the isolated and purified the compounds assay-1 through assay-10 was evaluated, and the results are shown in FIGS. 3a to 3c.

All samples except the insoluble assay-9 showed a concentration-dependent decrease in NO production within a non-cytotoxic concentration range (FIG. 3a). For Assay-5 and Assay-10, which showed relatively high NO reduction at the lowest concentrations, the expression levels of proinflammatory cytokines IL-1a and TNF-α mRNA, and PGE2 production measured by ELISA, were evaluated. Assay-5 showed a tendency toward decreased IL-1α mRNA expression with increasing concentration but did not inhibit TNF-α mRNA expression, while Assay-10 showed a tendency toward decreased expression of both IL-1α and TNF-α mRNA with increasing concentration (FIG. 3b). In addition, Assay-5 showed an increase in PGE2 production with increasing concentration, while Assay-10 showed a decrease in PGE2 production with increasing concentration (FIG. 3c).

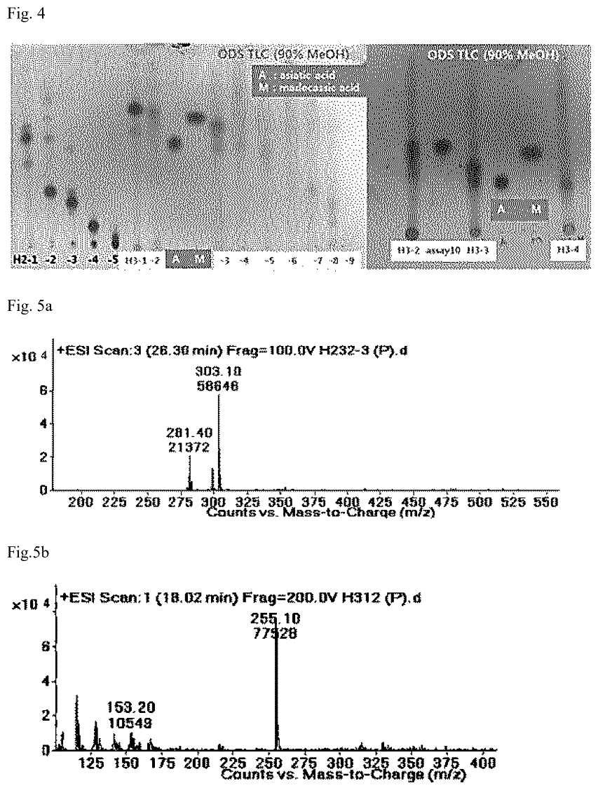

Therefore, anti-inflammatory efficacy was confirmed for both Assay-5 and Assay-10, while Assay-5 was predicted to have limited anti-inflammatory efficacy in terms of anti-inflammatory effect. These two samples were compared with asiatic acid and madecassic acid, well-known active ingredients in conventional Centella asiatica, by developing them on ODS TLC (developing solvent: 90% aqueous methanol). It was confirmed that neither Assay-5 nor Assay-10 corresponded to asiatic acid or madecassic acid (FIG. 4).

### Example 2. Structural Analysis and Identification of the Active Ingredients

To determine the molecular weights of Assay-5 and Assay-10, LC-ESI-mass was performed. As a result, Assay-5 exhibited a [M+Na]⁺ peak at m/z 303.1 and a [M+H]⁺ peak at m/z 281.4, confirming a molecular weight of 280 (FIG. 5a). Assay-10 exhibited a [M+Na]⁺ peak at m/z 255.1, confirming a molecular weight of 232 (FIG. 5b).

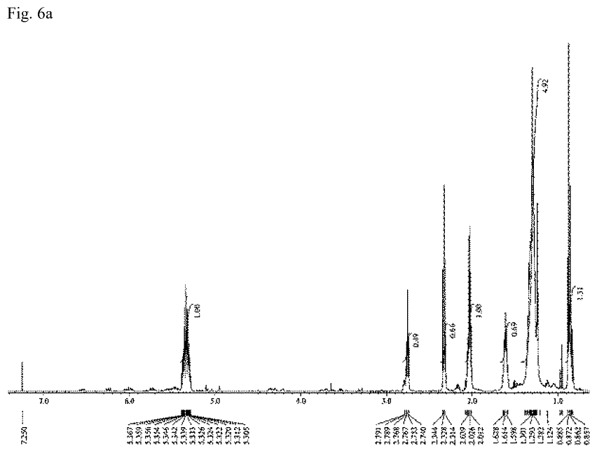

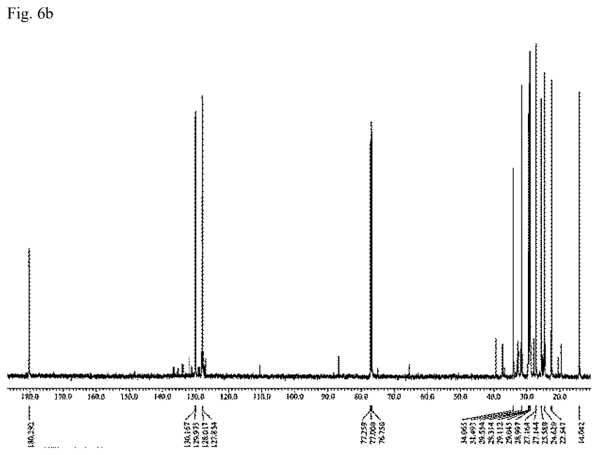

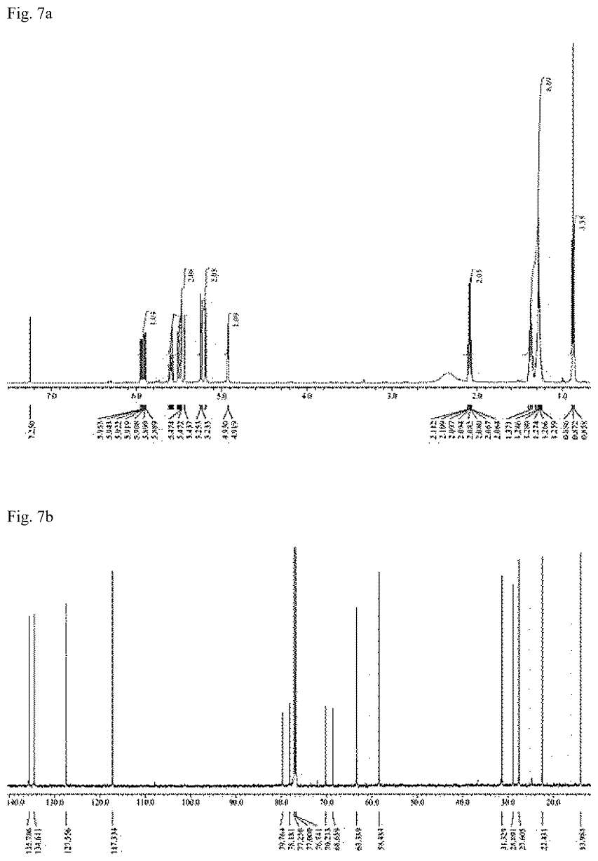

In addition, to elucidate the chemical structures of the two active ingredients, one-dimensional NMR spectra such as 1H NMR and 13C NMR, were measured after dissolving the samples in CDCl₃, and the results are shown in FIGS. 6 and 7.

As a result of measuring the 1H NMR spectrum of Assay-5, proton peaks corresponding to four olefinic protons attributed to two double bonds were observed between 5.3-5.4 ppm, one methylene proton was observed at 2.75 (t, J=6.8 Hz) ppm, one methylene proton was observed at 2.33 (t, J=7.5 Hz) ppm, two methylene protons were observed at 2.04 (m) ppm, one methylene proton was observed at 1.61 (m) ppm, seven methylene protons were observed between 1.2-1.4 ppm, and one proton peak originating from one methyl group were observed at 0.87 (t, J=7.0 Hz) ppm (FIG. 6a). Based on the 1H NMR spectrum described above, the present compound was predicted to be an unsaturated fatty acid composed of two double bonds, twelve methylene groups, and one methyl group.

As a result of measuring the 13C NMR spectrum of Assay-5, one carbonyl carbon originating from a carboxylic acid was observed at 180.3 ppm; four sp² methine carbons constituting two double bonds were observed at 130.2, 130.0, 128.0, and 127.9 ppm; twelve methylene carbons were observed at 34.1, 31.5, 29-30, 27.2, 27.1, 25.6, 24.6, and 22.5 ppm; and one methyl carbon was observed at 14.0 ppm (FIG. 6b). The 13C NMR spectrum was also consistent with the above 1H NMR spectrum, indicating that the compound is an unsaturated fatty acid composed of twelve methylene groups, two double bonds, and one methyl group.

As a result of measuring the 1H NMR spectrum of Assay-10, proton signals corresponding to three olefinic protons were observed at 5.92 (m), 5.60 (ddt, J=11.5, 0.5, 7.5 Hz), and 5.51 (ddt, J=11.5, 8.0, 1.5 Hz) ppm, one terminal methylene proton was observed at 5.45 (d, J=17.5 Hz)/5.24 (d, J=10.0 Hz) ppm, two oxygenated methine protons were observed at 5.19 (d, J=8.0 Hz) and 4.92 (d, J=5.5 Hz) ppm, four methylene protons were observed at 2.09 (m), 1.37 (m), 1.28 (m), and 1.26 (m) ppm, and one proton peak originating from one methyl group was observed at 0.87 (t, J=7.0 Hz) ppm (FIG. 7a).

As a result of measuring the 13C NMR spectrum of Assay-10, three sp² methine carbons were observed at 135.7, 134.6, and 127.6 ppm, one terminal methylene carbon was observed at 117.3 ppm, four quaternary carbons were observed at 79.8, 78.2, 70.2, and 68.7 ppm, two oxygenated methine carbons were observed at 63.4 and 58.5 ppm, four methylene carbons were observed at 31.3, 28.9, 27.6, and 22.4 ppm, and one methyl carbon was observed at 14.0 ppm (FIG. 7b).

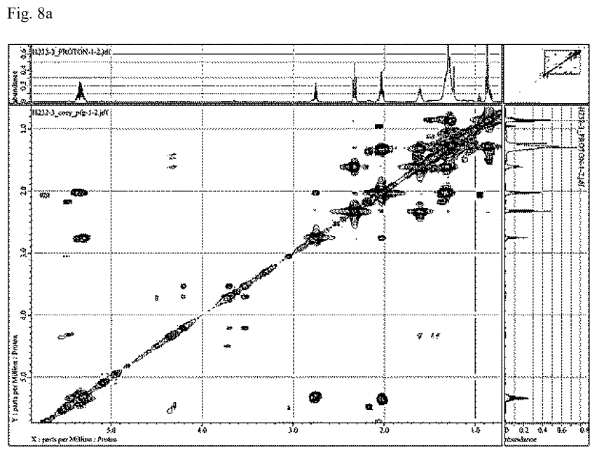

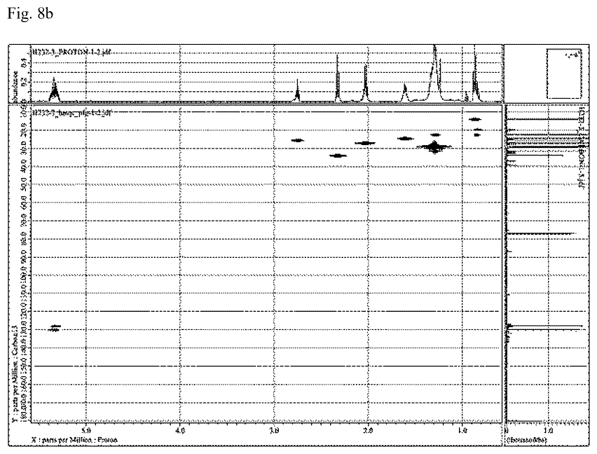

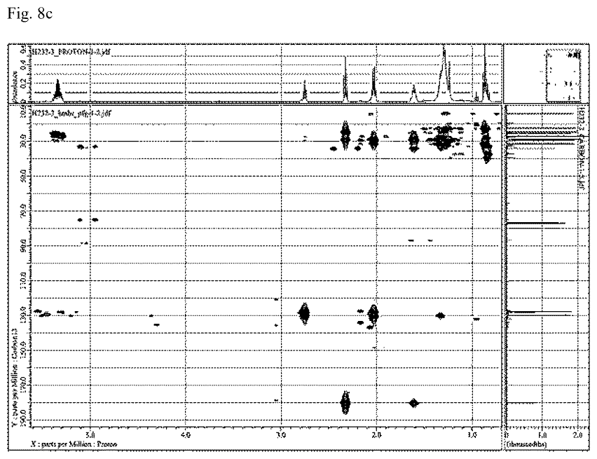

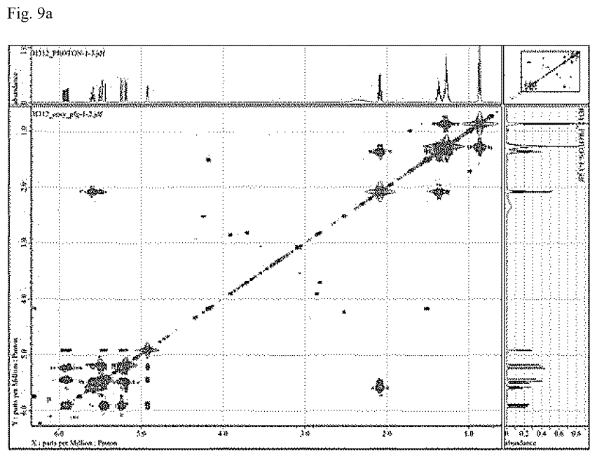

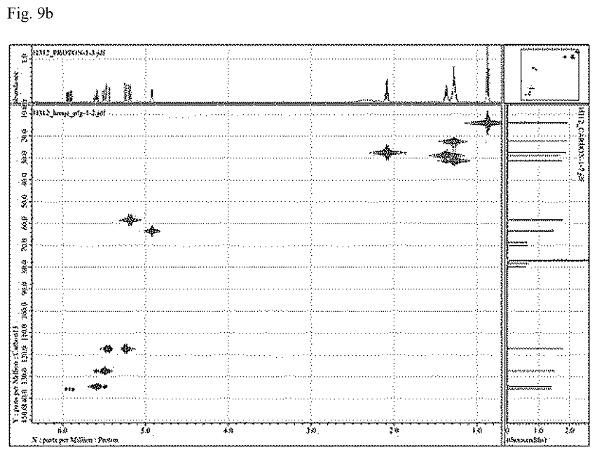

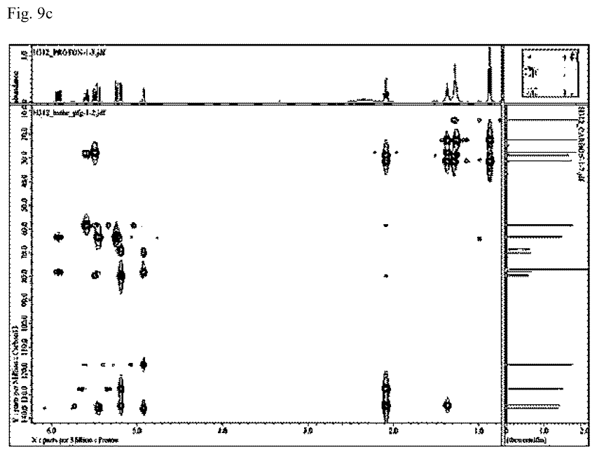

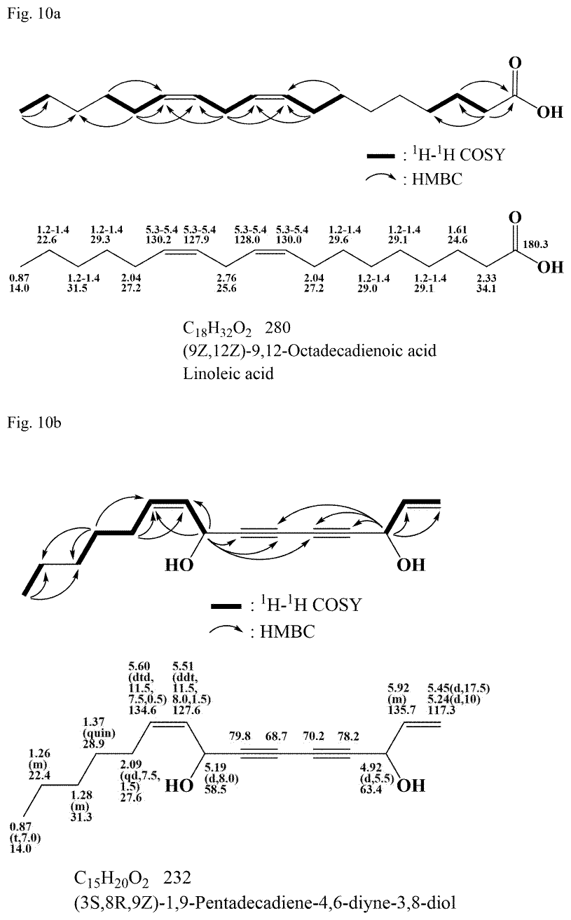

To elucidate the chemical structures of the two active ingredients, two-dimensional NMR spectra such as 1H-1H COSY, HMQC, and HMBC were measured to determine correlations between neighboring protons (3JH-H), one-bond correlations between protons and carbons (1JC-H), and two- or three-bond correlations between protons and carbons (2JC-H, 3JC-H), and the results are shown in FIGS. 8 and 9.

As a result, in the case of Assay-5, long-range correlations were observed from the methylene protons at 2.33 and 1.61 ppm to the carbonyl carbon at 180.3 ppm, and from the methylene proton at 2.04 ppm to the sp² methine carbons at 130.2, 130.0, 128.0, and 127.9 ppm. Additionally, long-range correlations were observed from the methylene proton at 2.04 ppm and the methyl proton at 0.87 ppm to the methylene carbon at 31.5 ppm, confirming the position of the double bonds. Although interpretation of the HMBC correlations was not possible due to overlapping methylene proton signals between 1.2 and 1.4 ppm and overlapping methylene carbon signals at 29.0-29.1 ppm, the compound was confirmed to be an unsaturated fatty acid composed of two double bonds and twelve methylene groups based on the preceding analysis. Furthermore, interpretation of the HMBC spectrum confirmed that the double bonds were located at the 9th and 12th carbon positions, respectively (FIGS. 8a to 8c).

In the case of Assay-10, long-range correlations were observed from the methyl proton at 0.87 ppm to the methylene carbons at 31.3 and 22.4 ppm, from the oxygenated methine proton at 5.19 ppm to the quaternary carbons at 79.8, 70.2, and 68.7 ppm, and from the oxygenated methine proton at 4.92 ppm to the quaternary carbons at 78.2, 70.2, and 68.7 ppm. Additionally, the carbon at 79.8, 78.2, 70.2, and 68.7 ppm were inferred to be carbons of a triple bond based on the molecular weight and the carbon chemical shifts (FIGS. 9a to 9c).

Accordingly, the chemical structures of Assay-5 and Assay-10 were determined as shown in FIGS. 8a and 8b, respectively. Based on the elucidated structures, a search of the SciFinder database identified the compounds as linoleic acid and araliadiol [(9Z)-1,9-pentadecadiene-4,6-diyne-3,8-diol], respectively.

In the experimental results of the embodiments of the present invention, when comparing anti-inflammatory efficacy at the same concentration, araliadiol (Assay-10) exhibited a higher NO reduction rate than linoleic acid (Assay-5), and also the expression levels of early inflammatory molecules IL-1α and TNF-α mRNA were also relatively more reduced by araliadiol compared to linoleic acid. Furthermore, linoleic acid increased the production of the inflammatory factor PGE2 as its concentration increased, whereas araliadiol decreased PGE2 production with increasing concentration. Therefore, based on the specific characteristics of the above-described markers, araliadiol is confirmed to exhibit stronger anti-inflammatory efficacy than linoleic acid.

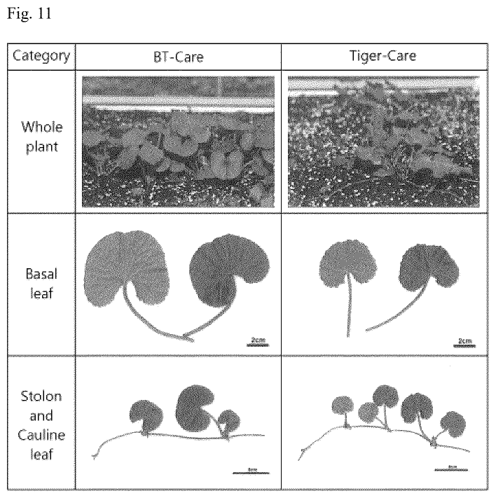

Araliadiol used in the embodiments of the present invention is a compound isolated from an extract of the Centella asiatica variety ‘BT-Care’. FIG. 11 is a comparison table of the whole plant and individual parts (basal leaf, stolon and cauline leaf) of the ‘BT-Care’ variety and a common variety (Tiger Care). As shown in FIG. 11, ‘BT-Care’ was confirmed to be a variety exhibiting greater length and width than the control variety ‘Tiger Care’ in each plant part, including the whole plant, basal leaves, stolons, and cauline leaves.

1. An anti-inflammatory composition comprising araliadiol or a pharmaceutically acceptable salt thereof as an active ingredient.

2. The anti-inflammatory composition according to claim 1,
wherein the concentration of the active ingredient is 0.00001 to 9% by weight, 0.0001 to 5% by weight, or 0.001 to 3% by weight, based on the total weight of the composition.

3. A method for preventing dermatitis and improving skin condition, comprising applying the composition of claim 1 to a subject's skin.

4. The method according to claim 3, wherein the composition is in any one form selected from the group consisting of solution, suspension, paste, gel, cream, lotion, powder, soap, surfactant-containing cleanser, oil, powder foundation, emulsion foundation, wax foundation, and spray.

5. A method for preventing or treating an inflammatory skin disease, comprising administering the composition of claim 1 to a subject in need thereof.

6. The method according to claim 5, wherein the inflammatory skin disease is selected from the group consisting of dermatitis, allergic dermatitis, irritant dermatitis, seborrheic dermatitis, atopic dermatitis, sensitive skin dermatitis, pruritus, eczematous skin disease, dry eczema, erythema, urticaria, psoriasis, drug eruption, acne, and vaginitis.

7. The method according to claim 5,
wherein the subject requires reducing at least one of NO production, PGE₂ production, IL-1α expression, and TNF-α expression.

8. A method for improving skin condition associated with inflammation, comprising consuming a food composition comprising the composition of claim 1.

9. The method according to claim 8, wherein the food composition is in the form of meat, sausage, bread, chocolate, candy, snacks, confectionery, pizza, ramen, noodles, chewing gum, dairy products, soup, beverages, tea drinks, alcoholic beverages, or vitamin complexes.


SEQUENCE LISTING (6 sequences)

SEQ ID NO: 1 - DNA, 20 bp, synthetic construct
  source 1..20: mol_type = other DNA
aacgaatttggtcgaacagc

SEQ ID NO: 2 - DNA, 17 bp, synthetic construct
  source 1..17: mol_type = other DNA
tgaggagggattcagtg

SEQ ID NO: 3 - DNA, 20 bp, synthetic construct
  source 1..20: mol_type = other DNA
tcaagatggccaaagttcct

SEQ ID NO: 4 - DNA, 21 bp, synthetic construct
  source 1..21: mol_type = other DNA
tgcaagtctcatgaagtgagc

SEQ ID NO: 5 - DNA, 20 bp, synthetic construct
  source 1..20: mol_type = other DNA
caaagtagacctgcccagac

SEQ ID NO: 6 - DNA, 20 bp, synthetic construct
  source 1..20: mol_type = other DNA
gacctctctctaatcagccc